(19) United States
(12) Patent Application Publication
(10) Pub. No.: US 2006/0224048 A1
(43) Pub. Date: Oct. 5, 2006

(54) Wearable personal area data network

(75) Inventors: Richard W. Devaul, Somerville, MA (US); Daniel Barkalow, Somerville, MA (US); John Carlton-Foss, Weston, MA (US); Christopher Elledge, Arlington, MA (US)

Correspondence Address: BERGMAN KUTA LLP, P. O. BOX 400167, CAMBRIDGE, MA, 02140, US

(73) Assignee: AWare Technologies, Inc.

(21) Appl. No.: 11/258,725

(22) Filed: Oct. 25, 2005

Related U.S. Application Data
(63) Continuation-in-part of application No. 11/121,799, filed on May 3, 2005.
(60) Provisional application No. 60/664,661, filed on Mar. 22, 2005.

(51) Int. Cl. A61B 5/00

(52) U.S. Cl. 600/300; 128/903

(57) ABSTRACT

A near-field communications network enables a body-wearable personal area network. The near-field communications takes place in the magnetic near-field. A master node with an orthogonal antenna array controls the network. The orthogonal antenna array is orientable to maximize signal strength. The antennas are designed and operated so as to strand the energy of transmission of communications in the near-field. This reduces the detectability of the communications outside of the immediate area of the near-field network.

### CROSS-REFERENCES

This application claims the benefit of U.S. provisional application Ser. No. 60/664,661 filed Mar. 22, 2005 and titled “Wireless Personal Area Network” by the present inventors, the disclosure of which is incorporated herein in the entirety.

This application is a continuation-in-part of U.S. patent application Ser. No. ______ filed Oct. 24, 2005 and entitled, “Method and System for Wearable Vital Signs and Physiology, Activity, and Environmental Monitoring” which is a continuation-in-part of U.S. patent application Ser. No. 11/121,799 filed May 3, 2005 and entitled, “Method and System for Wearable Vital Signs and Physiology, Activity, and Environmental Monitoring.”

### GOVERNMENT RIGHTS

This invention was made with Government support under contract W81XWH-04-C-0019 awarded by the United States Army and was derived from work partially funded by the Government under contract no. F33615-98-D-6000 from the Air Force Research Laboratory to Sytronics, Inc., and subcontract Sytronics P.O. no. 1173-9014-8001 by Sytronics to AKSI Solutions LLC. The Government retains certain rights in portions of the invention.

### BACKGROUND

The present invention relates generally to wireless personal area networks, and, more particularly, to such networks using inductive near-field data communications.

Typically, in conventional data communications, the object has been to increase the distance at which communications can be accomplished and to provide the largest possible bandwidth for data flow. There are, however, certain domains of data communications in which very different, if not seemingly contradictory, objectives are essential.

In military settings, for example, data communications among various microprocessors, sensors, and other devices on, in, or very near the body can enable important functionality for personnel in a field of operation, in the office, in medical facilities, and other venues. These communications would ideally be in the inductive, near-field electromagnetic regime which limits the ability of enemies to illicitly capture data to a few meters distance from such a communications device. The domain of detectability of the location of the device user is limited to no more than ten meters. This can be contrasted with RF data communications for which a datum can be intercepted at considerable distances and users can be located at a distance of one hundred kilometers, that is, by satellite.

In commercial office settings, for example, data communications among various electronic devices at characteristic distances of two meters, with possible extension by repeater devices, can serve as the basis for a personal or very small group area network. A cluster of personal computers, printers, scanners, sensors, microphones, PDA's, cell phones, and other devices can be located in a volume of approximately eight cubic meters. This constitutes a small neighborhood in which the data transmission requirements for wireless communications are relatively low in comparison to wired communications.

In public spaces such as busses and subways, for example, data communications among various electronic devices, such as a cell phones and PDA's, typically requires communication over a very short distance with a comparatively low bandwidth requirement for data transmission.

Another application for short distance communication involves communications systems where one or more of the communicating devices is located under water or immersed in certain other fluids or materials that allow inductive data communications but, typically, not RF data communications. Often these immersible devices need to be able to communicate data well whether immersed in fluid or not immersed.

In medical applications, wireless means are needed for communicating between devices such as implants located within the body and devices such as computers located outside or on the surface of the body. For example, there is a need for data sensors, such as heart or brain monitors, inside the body to be able to communicate with data receivers and computers outside the body for review by medical personnel. Further, it is desirable to have a means for providing information to adjust the function of an implanted medical device such as a pacemaker without resorting to wires or additional surgery. In this case, it is particularly desirable that the range of the data communications be quite short so that there is no interaction with other nearby data communications, and minimized interaction with noise or other electronic artifacts that may be produced within one hundred meters of the person.

It is further desirable to have two-way communications between the instances of components enabled with this technology. Even for pairings of such devices that seemingly provide for only one-way broadcast of information, such as a computer sending a document to a printer, or a sensor sending information to a receiver or a computer system, two-way communication is useful. Two-way communication is useful, for example, for error correction, efficient management of data resources, efficient use of energy resources through switching certain devices between sleep mode and active mode upon command, verification that a device is functioning properly, and for other reasons and functions.

One conventional technique that is used to establish wireless communications over short distances is Bluetooth, an open specification that is governed by the Bluetooth Special Interest Group (SIG). Bluetooth communication occurs in the unlicensed Industrial, Scientific, and Medical (ISM) band at 2.4 GHz. A Bluetooth transceiver uses spread-spectrum frequency hopping to reduce interference and fading. In this technique, a Bluetooth device uses seventy-nine individual, randomly chosen frequencies within a designated range, changing from one to another on a regular basis. Generally, the transmitter in a Bluetooth device changes frequencies 1,600 times every second.

Typically, a communications device using Bluetooth has a range of approximately ten meters. When Bluetooth-capable devices come within range of one another, an automatic electronic conversation takes place to determine whether they have data to share or whether one needs to control the other. Once the conversation has occurred, the Bluetooth devices form a network, referred to as a personal area network (PAN) or “piconet”. Once a piconet is established, the member devices randomly hop frequencies in unison so they stay in touch with one another and avoid other piconets that may be operating in the physical area. The devices in a piconet share a common communication data channel. The channel has a total capacity of 1 megabit per second (Mbps). Headers and handshaking information consume about 20 percent of this capacity. In the United States and Europe, the frequency range is 2,400 to 2,483.5 MHz, with 79 1-MHz radio frequency (RF) channels. In practice, the range is 2,402 MHz to 2,480 MHz. In Japan, the frequency range is 2,472 to 2,497 MHz with 23 1-MHz RF channels.

The MiniMitter device of Mini Mitter, Co. of Bend, Oreg. is a conventional device using inductive technology to communicate. The MiniMitter is a pill-sized device that the user swallows. Inside the human body, the pill senses the temperature in the digestive system and broadcasts this information to a receiver outside the body.

Another conventional device is the inductive electronic device developed by Aura Communications, Inc. of Wilmington, Mass. U.S. Pat. No. 6,459,882, assigned to Aura Communications, Inc., discloses a microchip system having three orthogonal transducers. The transducer system is connected to an antenna multiplexer electronics device.

Technical challenges in the implementation of wireless communications over short distances include establishing two-way communications between devices, error-detecting, error-correcting, collision-detection, collision-management and coordination of devices by a master controller.

For the foregoing reasons, there is a need for a system and method for wireless communications over short distances.

### SUMMARY

Embodiments of the present invention use the near-field magnetic component of the electromagnetic radiation spectrum to transmit signals and use induction for receiving the transmitted signals. The field strength of radiation in this region of the electromagnetic radiation spectrum drops off as the sixth power of the distance from the signal to the receiver which limiting the range of the data communications. The devices, accordingly, are able to establish a network for communications over close distances where the communications are also difficult to detect outside of the immediate area containing the devices.

The BlueTooth protocol serves as the basis for many manufacturers to construct and market radio frequency (“RF”) devices that provide ad hoc short-range wireless communications between or among a multiplicity of electronic devices. For example, one can use a BlueTooth mouse by Microsoft for operating one's computer, which can have a BlueTooth connection to a cell phone and a PDA. BlueTooth enabled devices thus are able form a short-range network, whether a very small wireless local area network or a personal area network. The BlueTooth protocol, however, uses radio frequency (RF) communications. RF signals are inherently non-local, thus allowing stand-off detection and also detection of the location of the user from great distances. Accordingly, the BlueTooth protocol inherently extends hundreds if not thousands of meters and does not support diminution or exclusion in public circumstances. In addition, the RF electronic signals in Bluetooth devices overlap and interfere with each other despite the frequency hopping. The overlap and interference create noise and other artifacts which in turn lead to equipment malfunction. The interference and noise between separate instances (i.e., separate BlueTooth networks) is difficult to block or eliminate.

The MiniMitter pill device is a primitive conventional approach to broadcasting and receiving data that uses inductive technology. Inside the human body, this pill senses the temperature in the person's digestive system and broadcasts this information to a receiver outside the person. The MinMitter pill has only one-way communication without error correction or detection.

Aura Communications has developed an inductive microchip. While this microchip allegedly supports data, its primary purpose is to support analog communications, and the corporation has historical refused to support efforts to develop a digital communications model. Thus, the Aura chip has been used as the basis for the Liberty Link headset for cell phones and radios. Currently Aura Communications has established a cooperation with Creative Solutions Inc. so that it can provide headsets to computer games that will not suffer interference from other nearby instances of such games with headsets. U.S. Pat. No. 6,459,882 discloses a system having three orthogonal transducers. The patent disclosure uses the term “transducer” instead of the more accurate term “antenna.” The transducer system is connected to an “antenna multiplexer electronics” device that manages the signal between the antennas/transducers and a transceiver. (The LibertyLink ASIC has x,y, and z antenna connections, which places the multiplexer inside the chip.) The specification does not disclose the workings of the signal management process. This patent specification discloses the notion of selecting one or more of the transducers for transmission and reception based on power consumption rather than effective orientation.

The inventive art in the present disclosure represents a significant improvement in that the present invention includes (a) two-way communication, (b) error-detection, (c) error-correction, (d) collision-detection, (e) collision-management, and (f) coordination by a master controller. The present invention is directed to an apparatus and method that implements a true personal area network as opposed to the conventional art that provides a broadcast and receive configuration similar to a broadcast radio station and a user. The present invention relates to magnetic inductive near-field data communications among such devices and entities as electronics, microelectronics, probes and censors, implants in humans and animals, devices that must communicate through such media as water that support communication using inductive near-field radiation but that do not support in particular communication using radio-frequency radiation, and devices that must communicate through the interface between such media and such other media as air and space.

According to one embodiment of the invention, an extended personal area data network includes a plurality of master devices receiving communications from slave devices. In one embodiment, the star topology of the wearable personal area data network is extended further with multi-master and master-to-master communications links.

According to one embodiment of the invention, a tiny, very light weight, unobtrusive, body-wearable personal area data network has an effective range of approximately 2 meters.

According to another embodiment of the invention, a tiny, very light weight, unobtrusive, body-wearable personal area data network has an effective range of approximately 2 meters. According to this embodiment, the personal area data network is not worn on the body, but is used with equipment or other components, whether all of this equipment is located within two meters of the central transceiver. A further arrangement of this embodiment includes means for extending the network through repeaters or other such devices.

In another embodiment of the invention, the personal area data network can transmit data and information through the human body and through the bodies of many other plants and animals, and therefore which can function both in the neighborhood of the human body, within the human body, and entirely within the human body.

In another embodiment of the invention, the personal area data network can transmit data and information through water and therefore which can function in underwater environments.

In another embodiment of the invention, the personal area data network is substantially undetectable at distances of greater than approximately 10 meters.

In another embodiment of the invention, the personal area data network communicates data in such a manner that unwanted parties can intercept that data at distances less than at most a few meters from the hub for the network.

In anther embodiment of the invention, the personal area data network communicates digital data, rather than just aural or analog information.

In another embodiment of the invention, the personal area data network includes bi-directional control and bi-direction communications.

In another embodiment of the invention, the personal area data network includes a separated or integrated control channel to manage communications and the availability of components and devices.

In another embodiment of the invention, one or more individual components and/or devices can be set into various modes of operation by electronic signals from the one or more control centers in the personal area data network.

In another embodiment of the invention, the personal area network controller includes means for setting components and devices into sleep mode, or an energy-conservation mode in order to reduce the rate at which power is used. This enables the personal area data network to conserve energy to extend battery lifetime.

Another embodiment of the invention includes means for keeping the generation of unwanted heat to a minimum thereby reducing waste heat energy to be dissipated into the nearby environment. This is particularly advantageous in that the nearby environment is often a human being.

In another embodiment of the invention, the personal area data network includes one or more hubs and one or more nodes but is small enough to be substantially unnoticeable by a wearer or user of the network. For example, the network includes hubs and nodes that are so small that they can be placed on a football player or other athlete in a full contact sport (other than boxing or martial arts), without posing a danger to the athlete in case of hard contact in the network area. In another example, the network hubs and nodes are able to be placed without notice on a soldier in the field.

In another embodiment of the invention, the personal area data network includes means to minimize and/or prevent crosstalk with components and devices that are parts of other different networks. In one arrangement, the network includes means to minimize and/or prevent crosstalk between components and devices of the network and other networks at distances of greater than 1 meter.

In another embodiment of the invention, the personal area data network has means to provide for communications between designated components and devices of the network and other networks.

In another embodiment of the invention, the personal area data network includes automatic registration of qualified transceivers and the sensors or other components whose data they communicate.

In another embodiment of the invention, the personal area data network includes means to provide error correction in the transmission of information.

In another embodiment of the invention, the personal area data network includes means to treat the three orthogonal antennae as an array or as an equivalent to a single optimally oriented virtual antenna so as to maximize the signal strength and bandwidth for communications within the network.

It is an object of the invention to provide a highly reliable device having a long battery life, so that both the user and potential others can rely on its information over a significantly long period of time.

It is another object of the invention to provide a device having very low power requirements in applications for which line power is available. Specifically, it is an object of the invention to function consistently with energy conservation guidelines.

It is another object of the invention to provide a device that avoids heat buildup.

It is another object of the invention to provide a device that is simple to use, preferably fully automated, so that users, or associated electronic gear, can attend to other tasks without cognitive or electronic burden demanded by the device.

It is another object of the invention to provide a device that is light and that has a small footprint so that the device does not add bulk or burden to any small electronic devices into which the present device is integrated, can readily be integrated into a user's clothing, can be attached to the body, or can be embedded within the body.

The present invention together with the above and other advantages may best be understood from the following detailed description of the embodiments of the invention illustrated in the drawings, wherein:

### DRAWINGS

FIG. 1 is a block diagram of a wireless personal area network according to principles of the invention;

FIG. 2 is a block diagram of a first configuration of a hub and sensor placement on a representative human figure according to principles of the invention;

FIG. 3 is a block diagram of a second configuration of a hub and sensor placement on a representative human figure according to principles of the invention;

FIG. 4 is a block diagram of a hub and sensor network according to principles of the invention; and

FIG. 5 is a block diagram of an extended personal area data network according to principles of the invention.

### DESCRIPTION

Embodiments of the present invention include a device that uses the near-field magnetic component of the electromagnetic radiation spectrum and inductive means for receiving said radiation. The magnetic field used as a transmission medium is modulated with a transmitter in which substantially all of the energy in the transmitting antenna coil is in the magnetic field. The field strength drops off as the sixth power of the distance from the signal to the receiver, severely limiting the range of the data communications making communications difficult to detect outside the immediate area of the network.

In the present application, the term “short range” is applied to wireless communications signals which, for reasons of the underlying physics and/or the specific details of the engineering application, attenuate more rapidly than RF propagating in free space, which is to say more rapidly than a factor of 1/r², where r is the distance from the transmitter. Examples of such “short range” communications modalities include: near-field inductive communications, near-field capacitive communications, body-coupled acoustic communications, UV free-space optics or other free-space optical communications using light frequencies rapidly scattered and attenuated by the atmosphere. A human body-coupled acoustic system relies on the impedance mismatch between the body and surrounding air to prevent signal (in this case, sound) leakage. In effect, the body itself acts as a wave-guide to confine the signal. Such modalities are distinguished from “non-short-range communications”, the category that includes all other wireless communications modalities, even so-called “short range RF” communications, which are attenuated proportionally to the square of the distance from the transmitter (or less rapidly, as in the case of planar RF waves).

By using a simple short range radio, the protocol can be handled on a lower power microcontroller. This reduces the space and power requirements from the 802.11 embodiment by not requiring a single board computer. In one embodiment, the low power telemonitor is a single unit of hardware constructed from three modules.

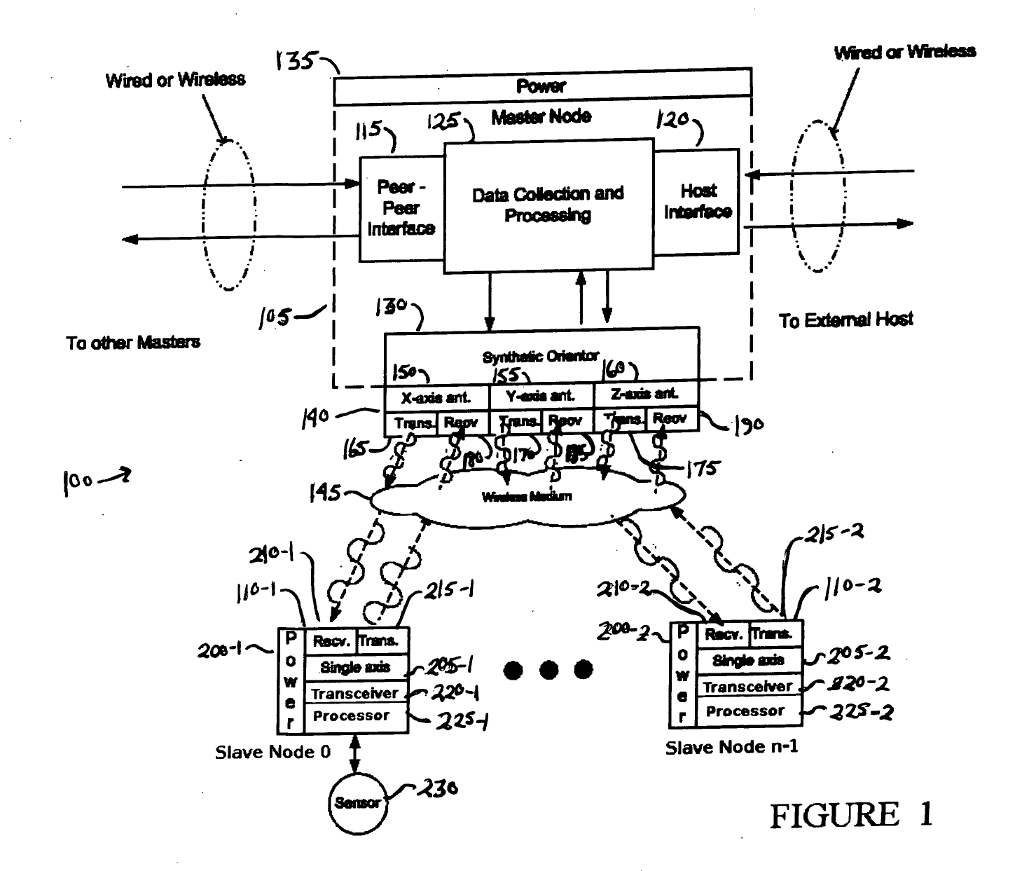

FIG. 1 is a block diagram of the functional components of a wireless personal area network (WPAN) 100 according to the present invention. The WPAN 100 includes a master node 105 and a plurality of slave nodes 110-1, 110-2 (collectively 110). The master node 105 has four major components, a peer-peer interface 115, a host interface 120, a data collection and processing component 125 and a synthetic orientor component 130. The synthetic orientor component 130 is in communication with an orthogonal antenna array 140.

WPAN Master Node

The peer-peer interface 115 enables peer-to-peer communication between the WPAN master node 105 and zero or more additional WPAN master nodes through either a wired communications medium (not shown) or a wireless communications medium 145. The wireless communications medium 145 is, for example, a near- or far-field RF medium, an UWB medium, an IR or UV optical medium, or an ultrasonic acoustic medium. In one embodiment of the invention, the wireless communications medium 145 is a magnetic field modulated by the synthetic orientor component 130 as described below.

The peer-peer interface 115 has two major functions. First, the peer-peer interface 115 provides an external host (not shown) a communications channel to talk to other external hosts which have WPAN master nodes. This allows the WPAN master node 105 to act as a general peer-to-peer network interface for communications between external hosts. One application of the WPAN 100 is to allow an external host to notify remote external hosts of important changes in status detected through sensors associated with one or more of the slave nodes 110.

The second major function of the peer-peer interface 115 is to allow an external host (not shown) to communicate with other WPAN master nodes (not shown). This feature allows several WPAN master nodes to operate as an extended WPAN network. If a remote external host is damaged or missing, the peer-peer interface 115 enables a local external host (not shown) to interrogate remote WPAN slave nodes (not shown) as though the remote slave nodes were part of the local WPAN 100.

The host interface 120 enables communication with zero or one external host (not shown). An example of an external host is a body-worn wearable data analysis or logging system. A WPAN master node 105 without an external host may still operate as part of an extended WPAN network, as described above. An external host may use the WPAN master host interface 120 for at least three types of communications. The first communications type is local WPAN network traffic, composed of commands and data sent from the external host to one or more WPAN slave node 110 in the local WPAN 100, and sensor data and other information sent back from local WPAN slave nodes 110 to the external host. The second communications type is WPAN master back-channel traffic, composed of commands and data sent from the external host to the local WPAN master node 105, and information sent from the WPAN master node 105 back to the external host. The third type of communications is WPAN extended network traffic, composed of commands and data sent from the external host through the peer-peer interface 115 to remote WPAN master nodes, and information sent back from remote external WPAN master nodes. This allows for the “extended network” operation if the remote WPAN master node is configured to allow interrogation of its network through the peer-peer interface 115.

The data collection and processing component 125 is, for example, a microprocessor or a microcontroller, that the WPAN master node 105 uses to implement the WPAN protocol, including such functions as scheduling slave nodes, talking through the communications interfaces, error correcting encoding/decoding, low-level encoding for transmission, and other data transaction control functions. The data collection and processing component 125 may also allow the WPAN master node 105 to perform real-time analysis of selected data that it receives from local WPAN slave nodes 110 or through the peer-peer interface 115. The result of this analysis may be provided to a local or remote external host in addition to, or in place of, the data itself. The data analysis functionality of the WPAN master node 105 may be critical to managing network bandwidth, especially over low-bandwidth peer-peer interface 115 or host interface 120. The analysis functionality, like other aspects of the WPAN master data collection and processing component 125, operates under the control of the local external host. If there is no external host, the data collection and processing component 125 functionality may be controlled by a remote external host connection through the peer-peer interface 115.

In one embodiment of the invention, the wireless medium 145 is a magnetic field modulated with a transmitter in which substantially all of the energy in the transmitting antenna coil is in the magnetic field. The associated electric field component is small and accordingly very little of the energy of transmission leaks into the far field of RF radiation. Typically, the near-field/far-field energy transition distance is below a noise floor. Specifically, the synthetic orientor component 130 is a transceiver that modulates the transmission of data (as encoded and directed by the WPAN master node 105) through the orthogonal antenna array 140 to maximize the strength of the near-field coupling between the WPAN master node 105 and a particular slave node 110. It performs this task by phase and amplitude modulation of the signal transmitted on each of the three antennas. The synthetic orientor 130 also performs the function of maximizing the coupling of the WPAN master antenna system 140 to slave nodes 110. This is accomplished, for example, by selective phase and amplitude filtering of the signal received from three antennas 150, 155, 160 in the orthogonal antenna array 140, or by implementing simultaneous independent reception on all three antennas 150, 155, 160.

Antenna Array

The orthogonal antenna array 140 includes three antennas, an x-axis antenna 150, a y-axis antenna 155 and a z-axis antenna 160. Each antenna 150, 155, 160 has a respective transceiver 165, 170, 175 and a respective receiver 180, 185, 190. Each antenna 150, 155, 160 is for example, a ferrite-core loop antenna with a separate transmission and reception antenna winding, and appropriate LC bandpass filtering.

Wireless Medium

The wireless medium 145 is, for example, a near-field communications medium. The wireless medium 145 is a quasi-static magnetic flux induced by transmitting signals from local antenna coil windings into remote antenna coil windings, concentrated by the ferrite cores of the antenna elements and passively amplified by the multiple turns of the loop antenna windings.

WPAN Master Power Module

The power module 135 for the WPAN master node 105 includes a power storage system, such as a rechargeable lithium-polymer battery, a consumable battery (such as zinc/air), or a fuel cell. Other types of power storage systems are contemplated within the scope of the invention. The present invention is not limited to the power storage systems listed here. The power module 135 further includes a high-efficiency power regulation system that can provide regulated power at appropriate voltages and with requisite current capacity. The power regulation system is configured so that it does not interfere with the operation of the near-field communications medium. Switching capacitive regulators, the use of toroidal inductors, or the use of high-frequency switching inductor power regulators may be required.

Slave Node

Each WPAN slave node 110 includes a power component 200-1, 200-2 similar to the power module 135 for the master node 105. The master node 105 and each type of slave node 110 may have different specific requirements for power, such as different operating voltages or overall power consumption, and the details of the power system may be optimized for each. The slave node 110 further includes a single-axis antenna component 205-1, 205-2, a receiver 210-1, 210-2 and a transmitter 215-1, 215-2 for the antenna 205-1, 205-2 a slave node transceiver component 220-1,220-2, and a processor 225-1, 225-2. The primary function performed by the slave node 110 is to act as a network interface for a local device, such as a sensor 230, to exchange data with the WPAN master node 105. The slave node 110 passes commands and configuration information received from the WPAN master node 105 to the local device, and relays sensor data and other information from the local device to the WPAN master node 105 at the request of the master node 105.

Slave Node Single Axis Antenna Component

The single-axis antenna component 205-1, 205-2 is substantially similar in magnetic and electrical properties to the single component antennas 150, 155, 160 of the WPAN master antenna array 140.

Slave Node Transceiver

The slave node transmits and receives data on the transmitting and receiving windings of the coil of the single-axis antenna 205-1, 205-2. In the preferred embodiment, because the slave node 110-1, 110-2 employs only a single antenna, maximizing the coupling strength is left to the WPAN master node 105, although the inventive material should not be thought of as limited by this approach. The slave node processor is responsible for implementing the WPAN near-field communications protocol for bidirectional communications with the WPAN master node 15.

Slave Node Processor

The slave node processor 225-1, 225-2 implements all levels of a slave node WPAN protocol (described below), which includes high-level functions such as packetizing local device data and responding to WPAN master node 105 commands and scheduling requirements, as well as error correcting encoding and decoding and low-level encoding data for transmission. The slave node processor component 225-1, 225-2 is also responsible for bidirectional communications through a wired medium with sensors or other local devices for which the slave node 110-1, 110-2 provides a WPAN network interface.

In addition to implementing the WPAN protocol and acting as a bidirectional communications bridge between the local device and the WPAN master node 105, the slave node processor 225-1, 225-2 may also (at the direction of the master node 105) perform some processing or analysis of the data received from the local device prior to transmission, and provide that processed information in addition to or in place of the original data.

The present embodiment of the invention includes one master node 105 and a plurality of slave nodes 110. This configuration can form a core unit. Various devices and networks can be built from the core unit by having a multiplicity of the units operating in series, in parallel, or recursively. In the current core embodiment, the master node 105 and slave nodes 110 use, for example, the Xemics XE1209 chipset, from the XEMICS Corporation of Switzerland, as a transceiver for wireless communication. Each node 105, 110 also has an Atmel AVR microcontroller, from the Atmel Corporation of San Jose, Calif., which handles all the aspects of the inductive wireless personal area network (“IWPAN”) protocol. Both types of devices use a pair of linear power regulators. One regulator outputs 2.85 Volts for all the logic and wireless communications. The other regulator outputs 5.0 Volts for the RS232 line level converters.

One embodiment of the slave device 110 has an Atmel AVR ATmega8 microcontroller, an XE1209, and a RS232 line level converter. This embodiment, for example measures 1.5″ wide by 1.4″ long by 0.5″ high. Alternative embodiments are smaller and lighter implemented as an Application Specific Integrated Circuit (“ASIC”). The slave node 110 uses an antenna module that plugs into the device to allow the use and testing of different antennas. The slave node 110 provides debugging information on an RS232 port and also receives data to be transferred to the master node 105 through the RS232 port. The slave node 110 uses 18 mA of current on the 2.85 Volt power line during normal operation. Each LED uses an additional 5 mA when active. Wireless transmissions pull a specified amount of current according to programming, varying from 1.8 mA to 110 mA in a square wave cycle. The RS232 converter uses between 2 mA and 10 mA of current on the 5.0 Volt power line depending on the load and speed of RS232 communications.

One embodiment of the master node 105 has an Atmel AVR ATmega64 microcontroller which can be used for many computational purposes including implementing logic and data processing that makes the system “smart” in addition to the XE1209's, and a RS232 line level converter. It connects to a separate orthogonal antenna array. The XE1209's allow the master to communicate on two channels simultaneously on each of the three antennas. In this embodiment, the master node measures 2.0″ wide by 2.0″ long by 0.8″ high. The master node can communicate with a host system through two separate RS232 ports. The master node uses 33 mA of current on the 2.85 Volt power line during normal operation. Each LED uses an additional 5 mA when active. Wireless transmissions pull a specified amount of current according to programming, varying from 1.8 mA to 110 mA in a square wave cycle. The RS232 converter uses between 2 mA and 10 mA of current on the 5.0 Volt power line depending on the load and speed of RS232 communications.

WPAN Orthogonal Antenna Array

One of the challenges for a body-worn or ad hoc inductive network is a high dependence of transmission on the relative orientations of the antenna and the transmitter. Since this orientation generally cannot be controlled or predicted, alternative technical approaches are implemented to assure that maximum or at least adequate transmission occurs among master nodes and slave nodes, master nodes and other master nodes, when required. For the sake of clarity, this challenge is discussed in relation to the present invention using an embodiment in which inductive communication takes place between one master node 105 and one slave node 110. It should be understood that the invention is not limited to these device but instead can enable such communication among a plurality of such devices.

The goal is to produce a coupling between a master transmitter 165, 170, 175 and the slave receiver 210. The properties of near-field induction are that the transmitter produces a signal at a particular angle (the particular angle varies by location, and is the angle of the field lines in the classic iron filings and magnet demonstration), and the strength of the received signal is proportional to the absolute value of the cosine of the angle between the field angle and the receiver antenna angle. This means that there is no signal received if the receiver antenna is orthogonal to the field lines at its location. The inventive system is designed and implemented to be sure that, regardless of the orientation of the receiver antenna, a signal is received.

On the master, there is an array of three orthogonal antennas 150, 155, 160. The behavior of multiple antennas 150, 155, 160 connected to the same signal is to give the same effect as a single antenna at an angle which is a linear combination of the real antennas, weighted by the portion of the signal going to each antenna. This means that, by changing the distribution of the signal to the antennas, any transmitter orientation can, in effect, be produced.

The next issue is choosing an effective orientation for the transmitter. Finding one that works optimally with the receiver is impractical, because the devices are likely to change orientation quickly and unpredictably as a normal course of events. Therefore, the inventive system instead cycles through a range of effective orientations. By choosing a suitable pattern, one can ensure that no orientation is consistently in worse coupling than 57%, and the time-average coupling is never worse than 33%.

In order for the cycling to work, it has to be slower than the carrier frequency, so that it doesn't interfere with the signal detection, and faster than the bit rate, such that each orientation will be used during each bit, so that the bit will be received. The basic idea of the pattern is to rotate within a cone which goes through the three orientations of the physical antennas 150, 155, 160. Although those skilled in the art will readily recognize that there are potentially many designs for this purpose, the current preferred design is to switch discretely between the three antennas 150, 155, 160, which effectively jump between these directions.

In order to receive a signal on the master node 105 from a slave 110, the master node 105 listens simultaneously on each of three receiver antennas 180, 185, 190 (which in the present embodiment of the invention are coils around the same cores as the transmitters), such that some master receiver 180, 185, 190 has at least a 57% coupling with the slave transmitter 215. By looking at the error features of the bit sequences, the correct data is determined.

One embodiment uses this virtual antenna approach for reception, superposing the signals from the various physical antennas 150, 155, 160 into the signal that would be transferred to a single antenna oriented parallel to the field lines of the transmission and therefore picking up the maximum signal possible. In another embodiment, the master node 105 determines whether one of the three antennas 150, 155, 160 appears to have received a signal and uses that by itself. Thus this embodiment merely waits for one of the antennas 150, 155, 160 to pick up a start sequence for a data transmission, and uses that antenna for the brief transmission of data. In yet another embodiment, the master node 105 reads inputs from all three antennas 150, 155, 160 and uses the input from the one antenna that provides the best results for an error correction/detection algorithm (to be described below). In another embodiment, the master node 105 reads inputs from all three antennas and uses the one or more antennas that provide the best results for a multi-variate function (to be described below) that defines the goodness of the connection. Those skilled in the art will recognize that other algorithms, including variations and combinations of the above, may be conceived and implemented for satisfactory reception of data within the scope of the present invention.

There are two steps to the scheme for transmission. The first is to use the set of orthogonal antennas 150, 155, 160 transmitting at the same time with different strengths to simulate a single antenna at some other orientation. The second is to use the fact that one can change the angle of the simulated antenna electronically to simulate an antenna which is sweeping through different orientations (and therefore coupling with different receiver angles) without having a moving part.

WPAN Protocol General Features

In the preferred embodiment, the control protocol is designed to be a “hotplug” configuration in which slave nodes 110 may join (and leave) the system 100 at any time. The master node 105 therefore does not initialize. The master node 105 simply starts without any other devices connected.

The master device 105, after start-up, performs a round-robin poll of devices in the system 100. The master node 105 transmits a message to indicate to each known device and to each unknown device when to transmit, for how long, and what to transmit in return. In addition, the master node 105 transmits an instruction to unknown devices to send its serial number. The master node 105 then retransmits the received serial number, if a serial number was received, as well as a bus identification that the master node 105 intends to assign to the device having that serial number. If multiple devices respond to the serial number transmission, the device with the best signal wins, is assigned a bus identification, and does not transmit as unknown in the next cycle.

Instructions to known slave nodes 110 include both a start time and a duration, as well as a command which specifies what data should be sent. In addition, an extension allows the master node 105 to transmit general data to a slave node 110.

The process is as follows:
The master node 105 starts looking for devices;
A slave node 110-1 responds;
The master node 105 continues looking for devices, asks slave node 110-1 for a description;
Another slave node 110-2 responds, and the first slave node 110-1 sends description;
The master node 105 continues looking for devices, asks the first slave node 110-1 for data;
The first slave 110-1 sends data; and
etc.

If the master node 105 does not get a response to a transmission, the master node 105 retries. There is a separate command for repeating the previous data from the command for sending only new data, so the slave node 110 can determine whether new data is requested. If the slave node 110 does not respond with data within a designated period of time, then the registration of the slave node 110 with the master node 105 times out. The slave node 110 must re-register when the slave node 110 again becomes available and ready to respond with data.

WPAN Packet Layer

In a first embodiment of the present invention, the packet is 70 bits; 44 are data, 16 are error correction, and 10 are transition assurance (TA).

The packet is laid out as follows:
[4 data][2 data][1 TA][2 data][4 data][1 TA]
[4 data][2 data][1 TA][2 data][4 data][1 TA]
[4 data][2 data][1 TA][2 data][4 data][1 TA]
[4 data][2 data][1 TA][2 data][4 error ][1 TA]
[4 error][2 error][1 TA][2 data][4 error][1 TA]

In one embodiment of the invention, each TA bit is the opposite of the previous bit. This allows for one TA bit every 6 bits, ensuring that there are never 8 of the same bit in a row, which is a device constraint of the Xemics microchip. The TA bits are additionally used for error detection, since a high number of cases where this bit is not the opposite of the previous bit indicates that there are, in general, errors (or there is not actually a transmission). The TA bits are otherwise ignored.

Continuing with the same embodiment of the invention, at the beginning of a data transmission is the following header:
101010101010101001110

This header appears only once per transmission, regardless of the number of packets sent. (The first 16 bits are a Xemics device requirement.). Different embodiments of the invention differ in the last four bits and whether each 7th bit before the last four bits is a TA bit. One embodiment of the invention produces the header sequence by adding opposite bits to a sequence without every 7th bit.

A second embodiment of the invention, an embodiment that meets Xemics 1209 microchip specifications has a protocol including the following parts: Preamble, Transition Assurance, Error Correction, Bad Packet Detection, and Framing.

The preamble in this second embodiment calls for 16 alternating bits at the beginning of a transmission to allow the receivers' bit synchronizers to align to the correct timing. This alternative sequence ends with an additional nybble of “1011” to signal the start of data. Thus the beginning of a transmission in this embodiment begins with a complete preamble of “10101010 10101010 1011”. This allows the receiver to recognize the first bit of valid data after the preamble as long as it sees at least the final “1011”.

WPAN Control Protocol

The wireless personal area network control protocol is used to arrange when each device in the system 100 transmits. The system 100 uses the control protocol to transfer messages reliably. While one embodiment of the invention use the Xemics chipset identified above, those skilled in the art will recognize that alternative embodiments can be designed and fabricated for chipsets with differing characteristics. The properties provided in the present embodiment using the Xemics chipset include the following:
Two communications channels which do not significantly interfere;
A radio device may use only one channel at a time;
A radio device may only send or receive at any one time;
Switching channels and direction is swift;
The quantum of error-corrected data which may be transferred is 44-55 bits; The master node 105 can distinguish from among the devices sending on a channel and can also distinguish that there is no device sending on a particular channel.

One channel is defined as the data channel. The data channel is used exclusively by sensors 230 or other slave devices 110 sending data to the master node 105 (in normal operation). The primary goal of the control protocol in the present embodiment is to arrange for slave devices 110 to send on the data channel as much as possible without interfering with each other. Those skilled in the art will recognize that other criteria could be used to establish other protocol configurations.

The master node 105 in the present embodiment has two or more radios, so that the master node 105 can interact on both channels at the same time. Slave nodes 110 have only one radio.

Master Serial Interface

Messages from the master node 105 have the general format of ID, length, data. The length is the number of bytes of data. The command “0xFF” is invalid as either ID or length, and is used to synchronize the connection when the master loses power and accidentally sends a byte. Additionally, the master node 105 pauses only between messages, so transferring at a rate slower than 9600 baud also indicates that the next character will start a new transmission. A message having an ID of 0 is known to have been formatted or created by the master node 105.

In messages from slave devices 110, if the ID is in the range 1-127, it is the bus ID (not shifted) of a device, and the data is data received from the device. At the beginning of the data section (included in the length) is a code which indicates what the message means.

Host Protocol

This is a protocol to be used by any device which connects on one side to a serial connection and on the other side to multiple devices. This protocol is one of the alternative protocols used by the WPAN master node 105 and is also used by the radio-lined devices in the network.

The host protocol assumes that either end of the created link may be reset at any time, but that no data will be lost otherwise, and that both ends are able to keep the connection saturated for a message length. Each of the multiple devices in the link has an ID assigned temporarily to it. The ID is optionally a 7-bit value (bus ID). The master node 105 assigns these IDs. The IDs are optionally used in communicating with the devices themselves. The IDs are used to identify which device the master node 105 is referring to.

Framing

The messages in the host protocol in the present embodiment have the following form:
Start of message: 0x05 0xFF 0x50
Length of message (1 byte, including opcode, not including length or start)
Opcode (1 byte)
(data).

Synchronization is done by waiting for either a substantial pause or a start sequence, followed by a length, followed by that many bytes, followed by another start sequence.

The messages sent from the host have the following protocol:
Downlink Message 0x01: Status Request
0x01
(optional ID).

The host may send a message requesting information about connected devices. The master node 105 responds by sending “device connected” messages for all devices which are connected (if an ID is not given) or the requested device (if an ID is given).

The downlink message is as follows: 0x02: Disconnect. This command causes the master node 105 to disconnect all connected devices. The downlink messages 0x06 to 0x3F are reserved for future use. These commands do not evoke direct responses. The downlink messages 0x40 to 0x7F send device-specific command with no response. The messages have the following format: (command); ID; (data). The meanings of these commands vary depending on the device to which they are sent, but they do not evoke direct responses. The downlink messages 0x80 to 0xBF are reserved for future use, but require IDs and are expected to result in a response with the same opcode and ID. The downlink messages 0xC0 to 0xFE are for device dependant with response and have the following format: (command); ID; (data). The meanings of these commands vary depending on the device they are sent to, but they are expected to evoke a response with the same opcode and ID. The message 0xFF is reserved.

Messages to the Host

Typically, all messages to the host start with the ID of the relevant device, unlike messages from the host, which may affect the network as a whole. The uplink message 0x01 means that the device is connected. The master node 105 may send a message reporting a connected device with the form:
0x01
new ID
device class and type code (2 bytes)
serial-number (6 bytes).

The uplink message 0x02 means device disconnected. The master node 105 may send a message reporting that a device has disconnected with the form:
0x02
ID

The uplink message 0x03 means data transfer and has the following format:
0x03
ID
(data).

The uplink message 0x06 means lost data. This command indicates that an expected packet from ID has been determined to have been lost. The message has the following format:
0x06
ID.

The uplink messages 0x80-0xFE are for command response and have the following format:
(command-opcode)
ID
(data).

These are responses to device-independant (0x80-0xBF) or device-dependant (0xC0-0xFE) commands.

The uplink command 0xFF is reserved.

In another embodiment of the invention, while one of the channels is defined as the data channel, the other channel is defined as the control channel. On the control channel, the master node 105 sends:
a bus id (7 bits)
1 (one bit)
a command (8 bits)
a max length (8 bits)
a start time (6 bits)
(14 unused bits)

The device with the specified bus id, slave node 110-1 for example, responds with a packet containing the following data:
a data length (8 bits, no more than the max length given)
a status (8 bits)
(28 unused bits)

If the data length was not 0, slave node 110-1 begins transmitting on the data channel at the specified start time. The master node 105, however, must confirm the reservation, because the slave node 110 is not allowed to use the window if the master node 105 did not receive the response correctly. If the master node 105 does not receive a correct transmission, the master node 105 treats the transmission as a failure result.

The master node 105 confirms the time reservation with a packet having the following data:
bus id (7 bits)
0 (one bit)
0x00 (8 bits)
length (8 bits)
start time (6 bits)
(14 unused bits)

From this point until a window after the end of the data transmission, the slave device 110-1 is in data transmit mode, and cannot be talked to.

Once the master node 105 has sent the confirmation, the master node 105 may begin negotiating with other slave nodes 110, even before the data window starts, because the master node 105 knows when the data window will end, and can arrange a new window after that. The master node 105 can also make transmissions for which the master node 105 does not expect a response from the slave nodes 110.

If the master node 105 is not allocating time at this point, the master node 105 sends a packet having the following data: 0 (44 bits) The transmission of this packet keeps the system 100 in synchronization.

Device Discovery

Each device 105, 110 in the system 100 has a unique 36-bit serial number. The master node 105 sends a variable-length mask, which indicates the initial/high-order bits of the serial numbers which match. The master node 105 does this by sending the mask, a 1, and 0s for the rest of the length of the packet. The position of the final 1 (which is not counted) dictates the length of the mask.

Specifically, the master node 105 sends out a packet having the following data to do device discovery in the system 100:
0x00 (7 bits)
mask (0-36 bits)
1 (1 bit)
0 (36-0 bits)

Each slave node 110 compares the mask with its serial number, and responds if there is a match. The slave node 110 responds to the device discovery packet with a packet having the following data:
checksum (8 bits)
serial number (36 bits)

If the master node 105 gets no responses to the device discovery packet, the master node 105 goes on to a next device.

If the master node 105 receives multiple responses to the device discovery packet, the master node 105 probes the region with a longer mask. The master node 105 is able to determine that multiple devices have responded because error correction will fail or a received checksum will not match. The master node 105, however, needs to identify the case where no device has responded.

If the master node 105 gets exactly one response, the master node 105 sends a packet having the following data:
bus id (7 bits)
1 (1 bit)
serial number (36 bits)

The device, slave node 110-1 for example, responds with the specified serial number begins to use the specified bus ID.

Device discovery can be done in the intervals in which the data channel has been allocated far enough in advance that the data channel is unlikely to fall idle when the data channel could be used effectively, and discovery can be suspended while the master node 105 negotiates with known devices. In fact, the usual case will be to do zero-length mask discovery periodically, so that if a single device is added at a time, the single device is easily found.

Device Deallocation

The master node 105 has a device time-out threshold. If the master 105 has not had cause to address a particular slave node 110 in that period, the master node 105 takes steps to deallocate the slave node 110. The master node 105 deallocates a bus ID of a particular device to be deallocated by not using the bus ID for the timeout period, at which point the device will automatically consider itself no longer to have an ID.

In order to avoid deallocation, a slave node 110 responds to a request from the master node 105 within the timeout period. In order to avoid deallocating devices in the system 100, the master node 105 initiates an interaction with every device 3 times within the timeout period so that all of the slave nodes 110 have a chance to stay connected even if there were 2 failed interactions. Other types of redundancy schemes are contemplated within the scope of the invention. The present invention is not limited to the redundancy scheme outlined above. In one embodiment of the invention, for example, the master node 105 will accept even responses with uncorrectable errors in keeping a slave device 110 allocated if the start sequence is present.

Timing

The timing control sequence for the system 100 is
[MA]X[SL]X[MB][MA]X[SL]X[MB] . . .

The master node 105 gives its proposal. There is a transition. A selected slave 110 gives its response. There is a second transition. The master node 105 gives its confirmation. The pattern then repeats. There is no transition between the master node's confirmation of the first cycle and the master node's proposal of the second cycle.

The data sequence proceeds as follows: [MA]X[SL]X[MB][MA]X[SL]X[MB] . . .

XData . . .

The selected slave 110 has the data channel that starts after a transition after the end of the master node's second section. The start time for a time allocation is the number of [MB][MA] sections that will pass before the window begins. The window ends after the slave node 110 has sent a preselected number of bytes (plus padding and error correction). The master node 105 arranges for the slave node 110 to have sent the preselected number of bytes before the transition between the current window and the next window.

Commands

Device-specific commands are in the range 0x80-0xFF, while generic commands are in the range 0x00-0x7F. The commands are used to manage such processes as the transfer of data from a slave node 110 to the master node 105. Slave nodes 110 generate data in some device-specific way, and then transfer it to the master node 105. Often the slave device 110 needs a command to tell it to acquire some data, and then upon receipt of the command, the slave node 110 acquires the data incrementally over a period of time. The slave node 110 finishes acquiring the requested information at some point that depends on such factors as the particular slave device and its situation. It is therefore useful, for example, to distinguish the case where there is more data coming but not yet available from the case where there is no more data coming.

The commands are as follows:
0x00: does nothing; allows the master node 105 to query the status of the slave node 110 without having any effect.
0x01: requests the next chunk of data, confirming any previous transfers.
0x00: the slave node 110 has no data to send at this time.
0x01: the slave node 110 has data of the specified size to send.
0x02: the slave node 110 is not in a mode which involves sending data.
0x03: the slave node 110 has data to send and will be done sending the data at the end of this transfer.
0x02: requests the data sent previously to be sent again.
0x00: the slave node 110 has no data to send at this time.
0x01: the slave node 110 has data of the specified size to send.
0x02: the slave node 110 is not in a mode which involves sending data.
0x03: the slave node 110 has data to send and will be done sending the data at the end of this transfer.
0x04: requests that the slave node 110 do something such that a person looking at a group of similar devices would be able to identify this one.
0x00: the slave node 110 is not capable of making itself distinctive.
0x01: the slave node 110 is making itself distinctive.
   Altenative WPAN Control Protocol

In an alternative WPAN control protocol, a device may receive transmissions on both channels at the same time, but may not receive on one channel while transmitting on the other channel. Preferably, the master node 105 determines whether it uses one channel or both channels. This protocol is on top of the same packet layer as in the first embodiment of the WPAN control protocol.

Requirements

In this alternative embodiment, a slave device 110 does not transmit unless it has received an instruction to do so. The slave nodes 110 have widely varying bandwidth requirements, from “report an event with no extra information every few minutes” to “50 bytes/second constantly”. It is therefore preferable to allocate bandwidth in periods of different length. It must be possible for the master node 105 to send a command to any device and get a short response, regardless of the bandwidth pattern the device uses. It should also be possible for a device to report a change in its bandwidth needs without being polled for that information.

Devices preferably should be able to distinguish a transmission from the master node 105 from any other transmission.

Properties

Devices (both the master and the slave) transmit data in 70-bit packets containing 44 bits of data. Each bit takes 550 microseconds. The start of a transmission takes 21 bits. A buffer period of 9 bits is used between when a transmission ends and when a device which got that transmission is expected to start the start sequence. That allows devices to read the packet and figure out how they should respond to it.

Design

There are two phases: the master phase and the slave phase. In the master phase, it sends instructions to all of the slaves. These instructions specify when the device should transmit, for how long, and on what channel. They also specify what the device should do to generate the data. Devices are addressed by position in the master phase request. The master phase consists of a single transmission on each channel (except that, if no devices are assigned to the non-default channel, the master might not actually transmit on it). This transmission has the following format:
0x9 [4 high bits]
Master ID [4 low bits]
Duration [8 bits] in packets

This transmission is recognizably different from the start of a slave transmission (which does not start with a 9). This transmission should also be different between one WPAN and another one, so that a device can tell if it has unexpectedly switched networks.

On the default channel, the master node 105 starts by doing a step in device discovery. If the master node 105 has received a message in the previous cycle, the master node 105 sends a message having the following format:
serial number: [24 bits]
bus id: [4 bits]

For each assigned device, the master node 105 sends a message having the following format:
command: [8 bits]
offset: [8 bits] in 100-bit periods
duration: [8 bits] in packets
channel: [4 bits]; 0 is channel 1, 1 is channel 2, other values TBA

If the command has the high bit set, the duration field is used for something different, and the duration is fixed at a single packet. In those cases where the channel is neither 0 nor 1, the offset and duration fields may have a different meaning.

Bus ID 0 is used for discovery. The odd bus IDs are addressed on the non-default channel. Thus, the default channel has the following format: (discovery), (id 2), (id 4), (id 6), etc.

After the master transmission phase, the devices respond. The slave phase begins after the master phase completes, as given by the duration at the start of the transmission. If the master node 105 is using both channels, the master node 105 gives the same duration for both.

The slave phase is divided into segments of 100 bits. This time is sufficient that a reaction, a start sequence, and a packet all fit. The durations and offsets are given in different units. A duration of 5 packets takes 30+70* 5=380 bits, which fits into 4 segments.

Each slave node 110 sends a message of the following format:
0xA: [4 bits]
response code: [4 low bits]
length: [8] in bytes

The slave node 110 then sends data. The slave node 110 gives the length in bytes, so the slave 110 can specify a length which is not an even number of packets.

The response code is used to respond to commands, and is also used, when transmitting data, to request a reconfiguration; this allows a device to change the size of its regular allocation. The maximum size which may be sent in an allocation is:

| packets | bytes |
|---|---|
| 1 | 3 |
| 2 | 9 |
| 3 | 14 |
| 4 | 20 |

Discovery

After a slave node 110 detects a master transmission, the slave node 110 sends in the reserved first segment on the default channel the following information, with response code being 0.

Commands

In this embodiment of the invention, the slave nodes 110 are initially unconfigured with respect to their time allocations. Therefore, the first step of the master node 105 is generally to ask the device for its information, including bandwidth needs.

Get Configuration

The request for information is as follows:
streaming data: [1 bit]
(unused): [7 bits]
bandwidth needs: [8 bits]

In various embodiments of the invention, a slave node 110 is ether a streaming slave, in which case the slave node 110 produces data at a constant rate, and recieves a regular bandwidth allocation, or the slave node 110 is event-based, in which case the slave node 110 will get a single packet each cycle (to report with), and will get more bandwidth (if configured) in the next cycle if it reports an event.

The system 100 will try to give a streaming slave a number of packets such that the slave node can fill the allocation with data and have less than a full packet left. The “bandwidth needs” field is in bytes/second.

The system 100 will give an event-based slave a single packet unless it reports an event, in which case it will, on the next cycle, give it the number of packets specified by the “bandwidth needs” field.

Get Data

Command 2 requests that the device send its data. The information is the slave's data. The response code is 0 normally; +1 if the slave has been forced to drop data (to stay up to date); +2 if the slave wants to change its allocation (in which case, the master's next command should be a 1).

Data Received in Response to a Get

Command 3 confirms receipt of the most recently sent data. The response code is 0. This uses the two-phase algorithm for reliable transport, since multiple confirmations of the same data have no extra effect, and multiple requests for data without a command 3 exchange get replays. (The master node 105 does a command 3 after each successful command 2, requiring a successful exchange before doing the next command 2.)

Identify Device

Command 4 requests that the device make itself visible to the user. This allows the user to identify which device is which when a number of similar devices are in use at the same time.

Error Rate

Command 5 requests that the device return its error rate on received data. It returns it in this format:
errors: [8 bits]
packets: [8 bits]

The device typically keeps these numbers in range by dividing both by the same factor on occasion (which keeps the rate the same, and causes older information to be less significant in the result).

Change ID

The master node 105 can change the ID of a device, so as to reduce the maximum id, which can make the master transmission shorter. The master node 105 sends command 0x10, with the new id in the “duration” field. The slave node 110 responds with a 0 status and no information. The master node 105 then uses the new ID. If the master node 105 does not receive a response, it still uses the new ID, but also sends the change ID command to the old ID, so that, if the slave node 110 did not receive the command previously, the slave node 110 will still be in synchronization with the master node 105. The master node 105 stops sending to the old ID when it either receives a suitable response to the command, or receives a response on the new ID (indicating that the slave node 110 received the message but the confirmation was lost).

Deallocation of IDs

The master node 105 deallocates a slave node 110 if the slave node 110 has not responded for 5 seconds. A slave node 110 considers itself deallocated if the slave node 110 has not received a master transmission for 5 seconds, if the slave node 110 receives a master transmission which would not be sufficiently long to address it, or if the slave node 110 receives a master transmission that has all 0s where the master transmission would address the slave node 110. The master node 105 should not reuse an ID, if possible, until the master node 105 has been unused (sending 0s or cutting it off) for 5 seconds.

Transition Assurance

The Xemics 1209 microchip calls for at least one transition from 0 to 1 or 1 to 0 every 8 bits to ensure that the receivers' bit synchronizers will stay aligned. The protocol can insure this by detecting runs of non-alternating bits. If 7 non-alternating bits are detected, an additional opposite bit is inserted. The receivers will simply ignore this bit when it detects the same condition.

In an alternative embodiment of the invention, instead of adding bits to avoid sequences, the master node 105 flips a bit if it would match the previous seven, and relies on error correction to repair it. A regular XOR mask is applied to the bit stream, so that the sequences of 8 matching bits are less common.

In a further alternative embodiment of the invention, a shorter XOR mask provides desirably transition properties. The selection of a shorter XOR mask makes use of a default transition-assurance mask having for example 16 alternating bits and the unencoded (for transition assurance) packet body.

Error Correction

A first alternative embodiment of the invention uses Hamming codes for error correction and a second alternative embodiment of the invention uses Reed-Solomon codes for error correction.

Hamming codes provide for forward error correction using a block parity mechanism. In general, Hamming codes allow the correction of single bit errors and detection of two bit errors per unit data, called a code word. This is accomplished by using more than one parity bit, each computed on different combination of bits in the data.

In the Hamming Code embodiment of the invention, the system 100 uses 4 interleaved streams of 11 bits of data, 4 bits of Hamming correction, and a parity bit for correction failure detection. This provides 44/64 data bits, corrects 4 consecutive errors, reliably detects 8 consecutive errors, corrects 1 error/16 bits in the long run, and detects 2 errors/16 bits in the long run.

The Reed-Solomon error correction scheme is a coding scheme which works by first constructing a polynomial from the data symbols to be transmitted and then sending an over-sampled plot of the polynomial instead of the original symbols themselves. Because of the redundant information contained in the over-sampled data, it is possible to reconstruct the original polynomial and thus the data symbols even in the face of transmission errors, up to a certain degree of error. Reed-Solomon Code is designed to work with bursts of errors which would probably be more likely to occur than single bit errors in a wireless link. Perhaps a short Reed-Solomon Code can be handled effectively by a microcontroller.

The embodiment of the invention that uses a Reed-Solomon Code includes, for example, blocks of 15 4-bit nibbles. Four of the nibbles used for error correction will correct all errors in 2 of the nibbles. The calculations necessary for Reed-Solomon error correction can be accomplished using a 256-byte table.

Bad Packet Detection

Some error correcting codes do not ensure that the data is valid after attempting to correct bits. A further embodiment of the Hamming Code embodiment includes a CRC error correction. A further alternative embodiment of the Reed-Solomon includes an ECC to detect bad packets.

Framing

In order to send variable length packets of data, a framing format is implemented for the packets. The following format, Preamble, ECC (Length, Data, Length, Data . . . , Length=0) enables a set of variable length data sets to be sent encapsulated by fixed length ECC blocks.

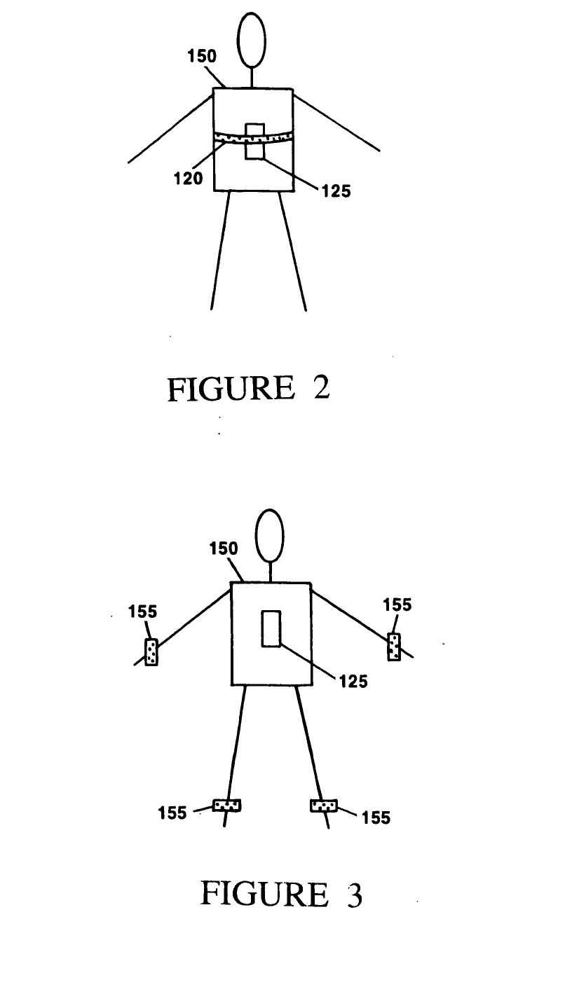

FIG. 2 is a block diagram of a first configuration of the hub and sensor placement on a human figure representation 150 according to principles of the invention. The human figure representation 150 is shown wearing a chest strap 120 having sensors (not shown) and a hub 125. The sensors include, for example, a piezoelectric breathing sensor and a polar heart monitor. The hub 125 includes, for example, an accelerometer and analytics. This example configuration of sensors can be used to monitor a patient with Parkinson's disease where pulmonary data, cardiovascular data and motion data are of interest.

FIG. 3 is a block diagram of a second configuration of the hub and sensor placement on a human figure representation 150 according to principles of the invention. The human figure representation 150 is shown wearing a hub 125 at the torso and sensors 155 at the wrists and ankles. The hub 125 includes, for example, an accelerometer and a wireless personal area network. The sensors are, for example, accelerometers and may include analytics. The sensors communicate wirelessly with the hub 125 through the wireless personal area network. In an alternative embodiment, the hub 125 and sensors 155 are included in a single on-body device.

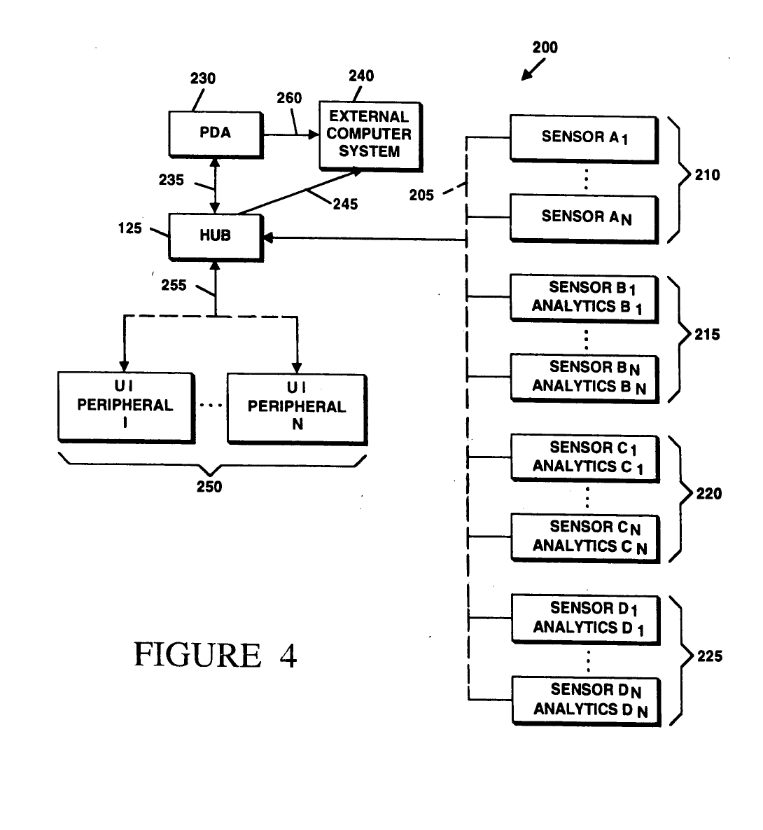

FIG. 4 is a block diagram of the hub and sensor network 200 according to the present invention. The hub and sensor network 200 includes a hub 125 connected through a first wired or a wireless personal area network (PAN) 205 a variety of sensors 210, 215, 220, 225. Sensors A 210 are without proactive communications abilities and instead are polled for data by the hub 125. Sensors B 215 are without proactive communications abilities however do include analytics. Sensors C 220 include both proactive communications and analytics. Sensors D 225 include proactive communications but are without analytics. One skilled in the art will understand that other types of sensors are within the scope of the present invention. The hub 125 is also connected to a PDA 230, or some other portable wireless communications device such as a cell phone, through a second wireless network 235. The hub 125 is further connected to an external local area network (LAN) or external computer system 240 through a wired or wireless connection 245. The hub 125 is still further connected to user interface peripherals 250 through a wired or wireless connection 255. The PDA 230 and external computer system 240 are connected through a wired or wireless connection 260. The communications linkages 235, 245, 255, 260 may be inductive, RF or may use some other communications modality. The type of communications modality used in a particular linkage 235, 245, 255, 260 depends on the distance the data needs to travel, the bandwidth needed, the communications medium (e.g., water, air) and other factors such as the need to prevent detection of other communications.

In operation, the hub 125 communicates with and controls the sensors 210, 215, 220, 225, directing the sensors 210, 215, 220, 225 to collect data and to transmit the collected data to the hub 125. Those sensors 220, 225 with proactive communications send collected data to the hub 125 under preselected conditions. The hub 125 also communicates with and controls the user interface peripherals 250. The hub 125 further communicates with portable devices such as the PDA 230 and with external network or computer systems 240. The hub 125 communicates data and data analysis to the peripherals 250, portable devices 230 and external systems 240.

The hub and sensor network 200 shown here is merely an example network. Alternative embodiments of the invention include a network 200 with fewer types of sensors, for example, including a network 200 with only one type of sensor. Further alternative embodiments include a network 200 with a hub 125 connected to only a PDA 230. In still further alternative embodiments, the various devices in the network 200 are able to communicate with each other without using the hub as an intermediary device. In short, many types of hub, sensor, communications devices, computer devices and peripheral devices are possible within the scope of the present invention. The present invention is not limited to those combinations of devices listed here.

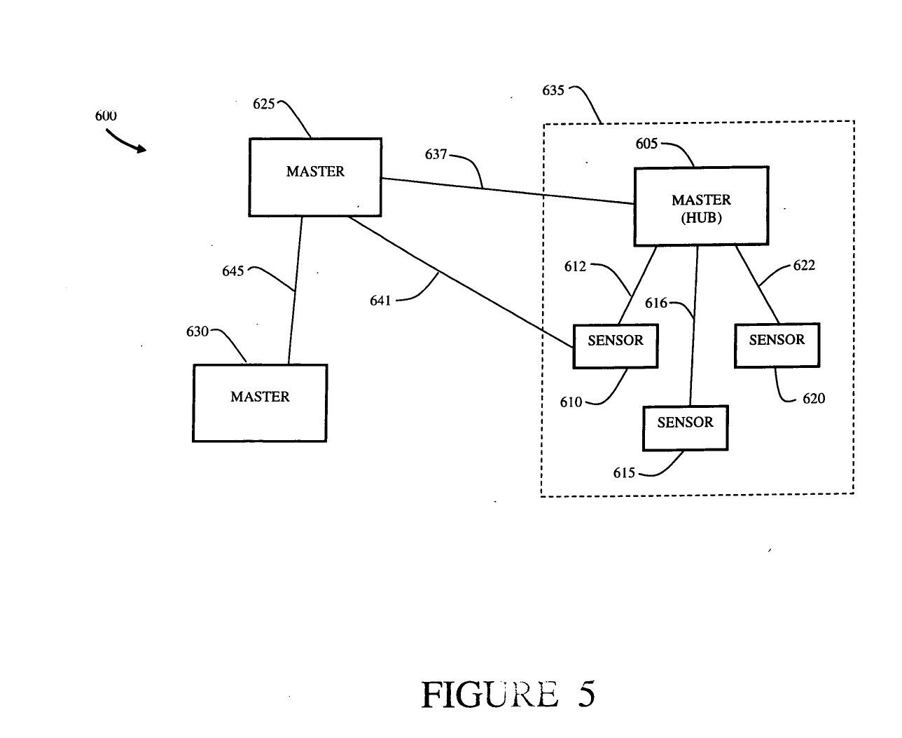

FIG. 5 shows an alternative embodiment of the wearable personal area data network (WPADN or “extended network”) described above. The network 600 shown in FIG. 5 is an extended personal area data network (XWPADN) which is a network that generally uses more than one communication modality and generally uses more than one “master” node.

Therefore, according to one embodiment of the invention, the network 600 includes three master nodes 605, 625, 640. A plurality of sensors 610, 615, 620 are connected to a first master 605 over communications links 612, 616, and 622 respectively. In this embodiment, the master 605 and sensors 610, 615, 620 are an on-body network 635 where the master 605 is similar to the hub 125 of FIGS. 2 and 3. Master 625 is in communication with master 605 over communications link 637 and with sensor 616 over communications link 641. Master 625 is in communication with master 630 over communications link 645.

In the present embodiment, the communications links 612, 616, 622 may be either wired or wireless links. Communications links 637, 641 in the present embodiment are wireless links while communications link 645 may be either wired or wireless. In alternative embodiments of the invention, the communications links may be some other combination of wired and wireless. As will be described below, a variety of protocols may be used over the communications links 612, 616, 622, 637, 641, 645.

The XWPADN protocol is generally implemented on top of the low-level link protocols provided by the underlying communications modalities. Typically, the XWPADN protocol is packet-based, employs multiple channels, and uses time domain multiple access (TDMA) channel sharing to allow multiple devices to share a small number of communications channels. The XWPADN protocol employs a star topology. In addition, embodiments of the extended network include a plurality of master devices as shown in FIG. 5. In these multi-master networks, a single slave device, such as a sensor, may report to more than one master, or two master devices may directly exchange information in a peer-to-peer network configuration. The extended network has further topological flexibility in that the star-topology network is extended with master-to-master communications links in addition to multimaster communications.

The extended network such as the network 600 of FIG. 5 uses mixed-mode short range (non-RF) wireless communications. The extended network includes the capability of using zero or more additional short-range wireless communications modalities, such as the combination of near-field inductive communications and UV communications, or more than one type of inductive near-field communications modes (e.g., a low-power, low-bandwidth modulation scheme with a higher-power, high-bandwidth).

Further, the extended network such as the network 600 uses mixed-mode short-range and non-short-range wireless communications. In some embodiments, short range, non-RF communications links are combined with RF systems, either to provide a longer-range communications capability in combination with the personal area network, to support legacy RF equipment or to provide increased bandwidth through the use of low-signature high-bandwidth RF communications techniques, such as pulse-shaped baseband modulation, otherwise known as Ultra-Wide Band (UWB). Use of other high bandwidth, low-signature RF communications techniques are considered to be within the scope of the present invention.

The extended network such as the network 600 combines short-range communications and passive or active wired (or other physical transmission media) extensions, on-body or off. For example, the use of a conventional wired digital communications (or mixed digital and analog) communications system for sensors integrated into a shirt or jacket using a short-range wireless communications link (such as inductive near-field) to communicate with another network in close proximity, such as a gear harness worn over the garment, the network in another garment, or a communications network in a vehicle, chair, etc.

The extended network such as the network 600 supports light-weight abstractions (and in some cases full implementations) of standard digital protocols, such as IP and TCP running over the XWPADN network. For some applications, the XWPADN functions in integration with other wireless or wired data networks. For example, XWPADN masters or XWPADN/IP network bridge nodes could perform network protocol translation and abstraction to make XWPADN master nodes appear as standard IP nodes. Support is not limited to IP and TCP protocols listed here. One skilled in the art will understand that the use of other standard wired and wireless protocols is possible within the scope of the invention. Further one skilled in the art will understand also that the use of higher-level protocols such as HTTP, the Enchantment IPC protocol, etc. is possible within the scope of the invention.

Topological Flexibility

The extended network such as the network 600 employs a star network topology so that a network master such as master 605 may manage the bandwidth allocated to each slave device, such as the sensors 610, 615, 620, discover new slaves, and generally manage the network in a centralized way. While this is a desirable network topology for many foreseeable applications of the WPADN, it is not the only useful network topology for short-range communications. Exceptions may include applications involving a mixture of on-body and off-body communications, or applications involving hybrid networks (combinations of XWPADN, and other types of short, medium, or long-range networks).

The extended network in some embodiments uses a protocol that supports zero or more masters communicating with a single slave, thus allowing “overlapping” short-range networks. This functionality is useful in a variety of situations. For example, one application of on-body short-range digital networks is physiology monitoring for soldiers. During routine operation, an on-body master node would interrogate body-worn slave nodes (such as sensors 610, 615, 620) to obtain physiology and activity information to determine health, metabolic load, etc. If, however, a soldier is injured a multi-master functionality would be important for combat casualty care. A multi-master capability would allow a medic or battlefield medical station to directly interrogate the soldier's physiology sensors (XWPADN slave nodes) regardless of the state of the soldier's own XWPADN master, which might be damaged or otherwise inaccessible. Another application for multi-master communications is a network in which a master node is integrated into a chair or vehicle, which would occasionally interrogate nearby slaves (such as the nodes being worn by the vehicle pilot or chair occupant) to determine whether such nodes required recharging, and to perform inductive charging of any nodes that required it.

Another feature of the XWPADN protocol according to certain embodiments of the present invention is direct bidirectional peer-to-peer communications between master nodes, such as master 625 and master 630 shown in FIG. 5, or (in some specialized situations) dedicated point-to-point bridge links. In master peer-to-peer mode, this configuration enables nearby masters to exchange information without the additional power drain involved in multi-master communications. For example, in the combat casualty care scenario described above, the medic could reduce the overall power drain on the soldier's physiology sensors by interrogating the soldier's master node, if that master node is still operational. The reason master-to-master communications burns less power than multi-master communications can be explained as follows: Slaves typically send the same type of information to both masters (in a two master network example). Further, slaves typically send on a similar communications duty cycle. As a result, a slave communicating with two masters tends to burn twice the power in transmission as a slave communicating with only one. If the same information is of interest to both masters, then one master may interrogate the slaves and then pass the information on to the other master. This saves power overall and shifts the increased power burden to a master node, which (since the master is necessarily transmitting more frequently than the slaves) is likely to have more spare power capacity.

A point-to-point bridge link is also foreseen as part of the XWPADN application. Such a link could be created between two masters, between a master and a dedicated XWPADN bridge node, or between two dedicated XWPADN bridge nodes. The use of a dedicated bridge node instead of a master may be desirable for some applications since a dedicated bridge node would implement only those protocol functions required for the network bridge and hence would be simpler and less expensive. The network bridge application is described in more detail below.

Mixed Mode Short Range Communications

The extended network includes a plurality of short-range communications modalities, generally a base modality and one or more other modalities. These additional modalities are, in some embodiments, as similar to the base modality as variations on the base modality near-field inductive modulation scheme that trade off increased bandwidth for power consumption. These modalities are, in some embodiments, as dissimilar as a combination of a body-coupled acoustic base modality, and an ultra violet (UV) free-space optics and capacitive near-field extended modalities. Other short-range communications modalities are considered to be within the scope of the present invention.

Multi-modal short-range communications are a desirable feature because no single short-range communications mode may be optimal for a given application. For example, one near-field inductive technology may have a low power cost for running a receiver, but a high power cost for running the transmitter. Likewise, another technology may provide higher bandwidth but at a significantly higher power cost for activating a receiver. A hybrid network combining the low-power base modality with the high-power extended modality could provide better power efficiency and higher bandwidth than either modality alone.

Mixed Mode Short-Range and Propagating RF Communications

For some personal area networking applications, it may be desirable to combine short-range communications modalities (such as inductive near-field) with RF communications, either to provide a necessary longer-range communications capability or to achieve higher bandwidth than can be supported by the short-range communications modalities or to support legacy RF systems. The XWPADN supports the use of RF for extended modalities, including the use of legacy 2.4 GHz and 5.8 GHz RF protocols such as Bluetooth and WiFi. The use of mixed mode short-range and RF communications decreases the benefits of using true short-range communications modalities, but there are situations which may call for both. One such situation is the combination of newer XWPADN gear with legacy RF systems. While a “network bridge” approach (see below) could be used to integrate two separate networks, in many applications it may be simpler and more efficient to support the RF network as an extended XWPADN communications modality. Another plausible scenario is the routine use of a true short-range base modality and the occasional use of a high-bandwidth RF extended modality, such as UWB, when the need for greater bandwidth outweighs the penalties in spectrum clutter and (for military or intelligence applications) the increased risk of standoff detection. Returning to the body-worn soldier physiology example previously discussed, it might be necessary for a medic to transfer a large quantity of medical history and/or recorded physiology data from a soldier's XWPADN network, and providing an option to do this quickly (at the cost of an increased RF signature) might be a valuable feature. In addition, the simultaneous use of a true short-range communications channel and a higher-power non-short range channel has security benefits, as the true short-range channel can be used to exchange authentication tokens and symmetric cryptography keys with little risk of eavesdropping, thus allowing highly secure communications on the non-short-range channel without the complexity and key-management and distribution problems of public key cryptography.

Combination of Short-Range Wireless and Passive or Active Wired Modalities.

The extended network uses, in some embodiments, wired (or physical medium channeled) communications first-class communications modalities. Both active and passive wired (or physical medium) modalities are used in embodiments of the present invention for short-range communications.

An active XWPADN wired communications modality is simply a conventional wired data bus employing XWPADN protocols. Such a wired bus is, in some embodiments, used to link several digital sensors together within the same body-worn garment. Zero or more active wired channels may be used in combination with wireless modalities in an XWPADN network. In some embodiments, the short-range wireless modalities are used to link between wired XWPADN segments. For example, a primarily wired XWPADN segment integrated into a shirt or jacket might communicate to a primarily wired XWPADN segment through a short-range wireless link located at the point of nearest overlap of the garments. By reducing the operational range of the wireless segment to the smallest possible distance, the power requirements for short-range wireless communication can be drastically reduced.

The passive use of physical media to channel short-range communications is also an important feature of the extended network of the present invention. An example of such is the use of body-coupled acoustic transducers for a body-area acoustic digital network. By using transducers with a significantly stronger coupling to the body than the surrounding air, the acoustic energy is primarily confined to the body itself, with minimal leakage into the surrounding air (the impedance mismatch at the body-air boundary serves to reflect the majority of the energy back into the body at the frequencies contemplated for this application.) In this case, the use of the passive physical medium is important to the operation of the communications modality. Physical media may also be used to extend and direct the reach of primarily wireless short-range signals, such as ferrous metal being used to extend and direct an inductive near-field wireless network. For example, specially designed ferrous elements in a patient-transport gurney might improve the coupling between the body-worn XWPADN sensor nodes of an injured soldier and a nearby medic's XWPADN.

Support for Existing Digital Network Protocols through XWPADN Networks

In many cases XWPADN systems will be required to operate in combination with other types of wired and wireless data networks. In such instances it may be desirable or important for these networks to exchange data. Embodiments of the extended network of the present invention support such interoperability and data exchange through the use of network bridging nodes and protocol translation.

A network bridging node is an XWPADN node with an additional “foreign” network interface. The job of the network bridging node is to bridge appropriate network traffic between the XWPADN network and the foreign network. For example, a network bridging node in the seat of a vehicle might be used to exchange data between the occupant's network and the vehicle's own digital network—perhaps allowing the occupant to use the vehicle's communications systems through the occupant's body-worn interfaces.

In order to exchange data between the XWPADN and the foreign network, typically the network bridging node provides appropriate data translation and abstraction for both networks. For example, if the foreign network is an IP (Internet Protocol) network, it will generally be necessary to for the network bridging node to provide the XWPADN network one or more effective IP addresses, and to abstract the various data sources on the XWPADN as either TCP or UDP sockets. Likewise data received from the IP network will be translated into the form of an XWPADN master-to-master or a slave-to-master communication. In general, this process may be described as a protocol abstraction, where the salient information from one network encapsulated in one protocol is abstracted from its specific protocol representation so that the information may be appropriately recoded in another protocol suitable for another network. To the extent to which there is no simple match in functionality or structure between XWPADN and foreign network protocols, it will be up to the network bridging node to provide whatever additional functionality and resources are required to perform the appropriate abstraction and translation. For example, TCP/IP packets may arrive out-of-order. The bridge node in this example needs to buffer and reassemble the contents of the packets in-order before handing off the contents to the XWPADN.

It is to be understood that the above-identified embodiments are simply illustrative of the principles of the invention. Various and other modifications and changes may be made by those skilled in the art which will embody the principles of the invention and fall within the spirit and scope thereof.

We claim:

1. A wearable master device for a remote monitoring network, comprising:
a controller to control the master device and to control data transmissions in the network;
a power module associated with the controller; and
a transceiver to receive data about a condition of a wearer of the wearable master device and to transmit the data about the condition to other devices in the network.

2. The wearable master device of claim 1 wherein the controller further comprises an analysis device to take the received data as input, the analysis device to determine the condition of the wearer in response to the received data.

3. The wearable master device of claim 1 wherein the transceiver further comprises a magnetic field generator, the magnetic field generator to generate a transmission medium through which the transceiver transmits and receives data.

4. The wearable master device of claim 3 wherein the transmission medium is a near-field RF field.

5. The wearable master device of claim 1 wherein the controller controls at least one wearable slave device.

6. The wearable master device of claim 5 wherein the wearable slave device is a sensor, the sensor to sense a condition of the wearer of the wearable master device.

7. The wearable master device of claim 1 wherein the transceiver transmits data to an external device.

8. The wearable master device of claim 7 wherein the external device is a second master device.

9. The wearable master device of claim 1 wherein the transceiver transmits data to a plurality of external devices.

10. The wearable master device of claim 9 wherein the plurality of external devices are master devices.

11. The wearable master device of claim 1 wherein the transceiver transmits data using a plurality of communications modalities.

12. The wearable master device of claim 3 wherein the magnetic field generator comprises at least one antenna.

13. The wearable master device of claim 3 wherein the magnetic field generator comprises an orthogonal antenna array.

14. The wearable master device of claim 13 wherein the orthogonal antenna array is configured to operate as a virtual single antenna.

15. The wearable master device of claim 3 wherein the magnetic field generator comprises an interface to other master devices.

16. The wearable master device of claim 3 wherein the transmission medium enables transmission through water.

17. The wearable master device of claim 3 wherein the transmission medium enables transmission through body fluids.

18. The wearable master device of claim 3 wherein the transmission medium enables transmission through living tissue.

19. A wearable personal area network, comprising:
a plurality of sensors to be placed on a wearer;
at least one analytic device to analyze data from at least one of the plurality of sensors, the analytic device to determine a condition of the wearer; and
a master device to control the plurality of sensors and the at least one analytic device, the master device to communicate the condition to at least one external device.

20. The wearable personal area network of claim 19 wherein one of the at least one external devices is a second master.

21. The wearable personal area network of claim 19 wherein one of the plurality of sensors communicates with one of the at least one external devices.

22. The wearable personal area network of claim 19 further comprising a near-field transmission medium over which the master device and the plurality of sensors communicate.

23. The wearable personal area network of claim 22 wherein the near-field transmission medium is a magnetic field.

24. A personal area data network, comprising:
a plurality of sensors placed on a person's body;
at least one analytic device to analyze data from at least one of the plurality of sensors, the analytic device to determine at least one condition of the person; and
a first master device to control the plurality of sensors and the at least one analytic device;
a second master device in communication with at least one of the first master device and the plurality of sensors; and
a third master device in communication with the second master device.

25. The personal area data network of claim 24 wherein the at least one analytic device further analyzes quality of the data from the at least one of the plurality of sensors.

26. The personal area data network of claim 24 wherein the second master device initiates communication with at least one of the plurality of sensors with the purpose of receiving sensor data.

27. The personal area data network of claim 24 wherein the second master device transmits the received sensor data to the third master.

28. A communications linkage, comprising:
a first master device;
a second master device; and
an inductive transmission medium to provide a communications medium enabling the first master device to communicate with the second master device.

29. The communications linkage of claim 28 wherein the inductive transmission medium further comprises a near-field RF field.
